(19) United States
(12) Patent Application Publication
(10) Pub. No.: US 2025/0035625 A1
(43) Pub. Date: Jan. 30, 2025

(54) Lateral Flow Assay Device and Method for Rapid Detection of Antibodies Against Felis Catus Gammaherpesvirus 1 in Domestic Cat Blood

(71) Applicant: BioVentures, LLC, Little Rock, AR (US)

(72) Inventors: Shana Owens, Little Rock, AR (US); James Forrest, Little Rock, AR (US)

(21) Appl. No.: 18/713,910

(22) PCT Filed: Nov. 28, 2022
(86) PCT No.: PCT/US22/51093

§ 371 Date: May 28, 2024

Related U.S. Application Data
(60) Provisional application No. 63/283,686, filed on Nov. 29, 2021.

(51) Int. Cl. G01N 33/543; G01N 33/569

(52) U.S. Cl. CPC G01N 33/54388; G01N 33/56994; G01N 2333/03; G01N 2469/20

(57) ABSTRACT

A lateral flow assay device for detection of Felis catus gammaherpesvirus 1 (FcaGHV-1) including a cassette having a sample well for receiving a drop of blood or serum from a patient cat. a testing well for displaying the result of the assay. and a control well for displaying the validity of the assay. A conjugate release pad is positioned in the cassette and includes gold nanoparticle-conjugated anti-feline IgG antibodies for binding IgG antibodies in the drop of blood or serum from the patient cat. A membrane is also positioned in the cassette and includes a test region and a control region. The test region includes a plurality of FcaGHV-1 antigens for binding antibodies against FcaGHV-1 antigens. and the control region includes Protein A proteins for binding feline IgG antibodies.

### CROSS-REFERENCE TO RELATED APPLICATIONS

This application claims the benefit of U.S. Provisional Application No. 63/283,686, entitled “Lateral Flow Assay for Rapid Detection of Antibodies Against Felis Catus Gammaherpesvirus 1 in Domestic Cat Blood” and filed on Nov. 29, 2021. The complete disclosure of said provisional application is hereby incorporated by reference.

### STATEMENT REGARDING FEDERALLY SPONSORED RESEARCH OR DEVELOPMENT

This invention was made with government support from grant no. 5TL1TR003109-02 awarded by the National Center for Advancing Translational Sciences. The government has certain rights in the invention.

### BACKGROUND

Gammaherpesviruses (GHVs) are DNA tumor viruses that establish lifelong chronic infections in lymphocytes, specifically B and T cells. Infection by human GHVs such as Epstein-Barr virus (EBV) and Kaposi sarcoma virus (KSHV) place individuals at risk of developing cancers, including Burkitt lymphoma, Hodgkin lymphoma, primary effusion lymphoma, Kaposi sarcoma, and many others. Nearly all adults are infected with one or both of these cancer-causing viruses. Felis catus gammaherpesvirus 1 (FcaGHV-1) is a feline GHV that was discovered in 2014.¹ FcaGHV1 infection is pan-lymphotropic, infecting both B and T lymphocyte, with the highest viral burden in the small intestines and bone marrow.² FcaGHV1 is frequently detected in domestic cats across the globe, including Australia, Singapore, Japan, and the USA.^{1,3} An estimated 30% of cats in the US are infected with FcaGHV1.^{1,4} Epidemiology data suggest that FcaGHV1 is a pathogen, as infected cats are about 3 times more likely to be classified as sick in blinded veterinary examinations.³ Like other GHVs, FcaGHV1 viral DNA is shed in oronasal fluids. These data indicate that viral DNA can be transmitted through saliva during natural behaviors such as grooming and biting.⁵ Like human GHVs, FcaGHV1 infection places domestic cats at risk of developing lymphoma.^{3,6} Feline lymphoma has been highly associated with retrovirus infection historically. Feline immunodeficiency virus (FIV) and feline leukemia virus (FeLV) infection increase the risk of lymphoma development by 6-to 60-fold, respectively. Mass testing, quarantining, and vaccination programs initiated in the 1970-80s led to a dramatic decrease in retroviral infection and subsequent disease.⁷ While the incidence of other forms of lymphoma, such as retrovirus-associated mediastinal lymphoma, has decreased in the past 40 years, GI lymphoma incidence has increased 50%.⁷ GI lymphoma is the most common malignancy diagnosed in cats, comprising 50-75% of all lymphomas.⁷⁻⁹ Feline GI lymphoma has a poor prognosis, with an average survival time of 4 months post-diagnosis.^{8,10} In situ hybridization revealed accumulation of FcaGHV1 genomes within gastrointestinal lymphomas, which suggests a potential role for FcaGHV1 in lymphomagenesis.¹¹ Together, these data suggest that FcaGHV1 infection is a co-factor for malignant transformation in domestic cats. Veterinarians are desperately seeking new tools to diagnose and therapies to treat or prevent this increasingly pervasive cancer in cats.

Currently, the only serology test available for FcaGHV1 is an enzyme-linked immunosorbent assay (ELISA), but this is not accessible for use in a veterinary setting. While highly sensitive, ELISAs require costly equipment with hours of setup and experimental time. It would therefore be desirable to develop a low cost, equipment-free, easy to use, rapid test for detecting the presence of antibodies against FcaGHV1.

These and other features, objects and advantages of the present invention will become better understood from a consideration of the following detailed description of the preferred embodiments and appended claims in conjunction with the drawings as described following:

### DISCLOSURE OF THE INVENTION

The present invention is directed to a lateral flow assay device for detection of Felis catus gammaherpesvirus 1 (FcaGHV-1), comprising:
a cassette, wherein the cassette comprises a sample well for receiving a drop of blood or serum from a patient cat, wherein the cassette further comprises a testing well and a control well, wherein a result of an assay is displayable in the testing well and a validity of an assay is displayable in the control well;
a conjugate release pad positioned in the cassette, wherein the conjugate release pad comprises a plurality of gold nanoparticle-conjugated anti-feline IgG antibodies for binding IgG antibodies in the drop of blood or serum from the patient cat;
a membrane positioned in the cassette having a test region and a control region, wherein the test region of the membrane comprises a plurality of FcaGHV-1 antigens for binding antibodies against FcaGHV-1 antigens, wherein the control region of the membrane comprises a plurality of Protein A proteins for binding feline IgG antibodies.

The present invention is also directed to a method of detecting Felis catus gammaherpesvirus 1 (FcaGHV-1) using a lateral flow assay device, comprising the steps of:
supplying a lateral flow assay device comprising a cassette, wherein the cassette comprises a sample well, a testing well, and a control well, wherein a conjugate release pad is positioned in the cassette, wherein the conjugate release pad comprises a plurality of gold nanoparticle-conjugated anti-feline IgG antibodies, wherein a membrane is positioned in the cassette having a test region and a control region, wherein the test region of the membrane comprises a plurality of FcaGHV-1 antigens, wherein the control region of the membrane comprises a plurality of Protein A proteins;
introducing a sample of blood or serum of a patient cat to the sample well of said cassette;
introducing phosphate buffered saline to the sample well to dilute the sample of blood or serum;
binding a plurality of IgG antibodies from the sample of blood or serum to at least some of the plurality of gold nanoparticle-conjugated anti-feline IgG antibodies on the conjugate release pad to form a plurality of complexes;

binding at least some of the plurality of complexes to at least some of the plurality of FcaGHV-1 antigens to display a positive result in said testing well; and
binding at least some of said plurality of complexes to at least some of the Protein A proteins to display a valid result in the control well.

### BRIEF DESCRIPTION OF THE DRAWINGS

FIG. 1 is a schematic of the lateral flow assay device of the present invention and the step-by-step process for using the lateral flow assay device of the present invention.

FIG. 2 is a schematic of an alternative embodiment of the lateral flow assay device of the present invention for detecting FcaGHV1, FeLV, and FIV, and the step-by-step process for using the lateral flow assay device of the present invention.

FIG. 3A is a western blot of FcaGHV1 viral antigens derived from a eukaryotic expression system that are used in the assay device. This system is necessary for production of modified (glycosylated) proteins, such as FcaGHV1 ORF8, that cannot be generated in bacteria.

FIG. 3B is a western blot of FcaGHV1 viral antigens derived from a bacterial expression system that are used in the assay device. Bacterial expression systems are more efficient than eukaryotic systems but are limited to production of proteins that do not require post-translation modifications such as glycosylation.

### DETAILED DESCRIPTION OF THE INVENTION

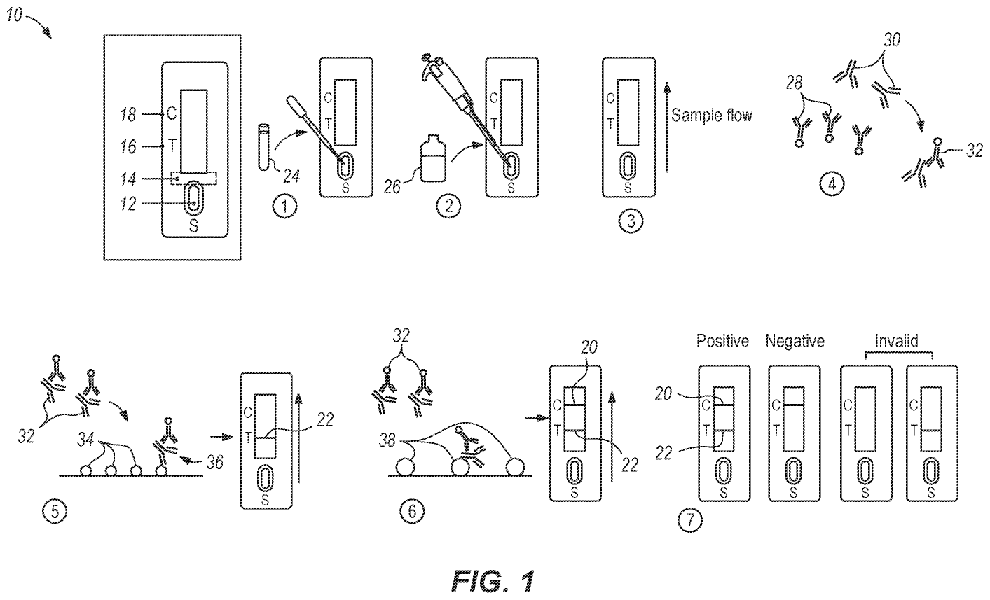

With reference to FIGS. 1-3B, the preferred embodiments of the present invention may be described. The present invention is directed to a lateral flow assay (LFA) device for detecting the presence of antibodies against FcaGHV1 and a method of using the LFA device to detect the present of antibodies against FcaGHV1. A lateral flow assay is a low-cost and equipment-free immunochromatographic assay that rapidly detects the presence of a target substance, such as an antibody, within a liquid sample.

The lateral flow assay device 10 of the present invention includes a sample well 12, a conjugate pad 14, a testing well 16, and a control well 18. The device 10 preferably is a plastic cassette that contains a glass-fiber sample pad, polyester conjugate release pad, nitrocellulose membrane, and cellulose absorbent pad of the type that would be well-known to a person of ordinary skill in the art. The conjugate release pad is the platform for the detection conjugate (gold-nanoparticle conjugated anti-feline IgG (AuNP-IgG) antibodies). On the nitrocellulose membrane, viral antigens 34 and control antigens 38 are immobilized for antibody detection. Beyond the membrane, the hydrophilic cellulose pad promotes capillary flow and absorbs excess sample. For proper capillary flow, the sample pad, conjugate release pad, and membrane must overlap. The sample pad extends over the conjugate release pad, which overlaps the nitrocellulose membrane. At the end of the nitrocellulose membrane, the wicking absorbent pad is layered on top to facilitate efficient sample flow.

In the present lateral flow assay design, a membrane is prepared with two test lines. The first test line 20 in the control well 18 is a control to confirm the presence of antibody within the blood sample. The test line 20 consists of protein A, a bacterial surface protein that binds immunoglobulins, or mouse anti-feline IgG antibody. The second test line is a test line 22 in the testing well 16 that contains purified antigens from FcaGHV1 (gene products from open reading frame (ORF) ORF8, ORF21, ORF38, ORF52, ORF73, and/or other surface glycoproteins) and is designed to capture antibodies that develop due to the presence of FcaGHV1 infection. Domestic cat blood or serum 24 (the analyte) will be applied to a sample pad where it will mix with goat anti-feline IgG antibodies conjugated to 40 nM gold nanoparticles (visually red). The analyte/antibody mixture will undergo capillary flow across the membrane. If antibodies against FcaGHV1 viral antigens exist in the sample, they will bind to the test line 22 and feline IgG antibodies will bind to the control protein A line 20. The gold-conjugated antibodies (AuNP-IgG) will appear as a red line to indicate a positive result. A positive result will bind both the test line 22 and control line 20, and a negative result will only have a red control line. An invalid test will be blank or only have a red test line 22.

As shown in FIG. 1, the process for using the lateral flow assay includes 7 steps. The sample is loaded in step 1. A drop of blood or serum 24 (preferably about 50 ul) from the cat being tested is applied to the sample well(S) 12 with a micropipette. Buffer 26 is loaded during step 2, in which phosphate buffered saline (PBS) is added to the sample well 12 to dilute the blood sample and promote capillary action through the diagnostic device 10. In the sample incubation step (step 3), the sample and PBS enters the glass fiber sample pad 14 where red blood cells are caught and the serum 24 rehydrates the polyester conjugate release pad. Capillary action moves the sample across the lateral flow test. In Step 4, AuNP-IgG antibodies 28 bind to the IgG antibodies 30 from the sample analyte within the polyester conjugate release pad. Due to capillary action, the analyte/AuNP-IgG complex 32 next enters the testing well 16 and crosses immobilized FcaGHV1 antigens 34 (encoded by FcaGHV1 ORF8, ORF21, ORF38, ORF52, & ORF73). In this FcaGHV1 antigen detection step (step 5), if the feline antibodies 30 recognize viral antigens 34, they will bind and form a visual complex 36. When that happens, a red line appears on the nitrocellulose membrane in the testing well 16. In the control antibody detection step (step 6), non-specific antibodies 32 continue to flow across the membrane and cross immobilized Protein A 38 or murine anti-feline IgG. Antibody complexes appear as a red line on the nitrocellulose membrane in the control well 18. In the final step (step 7), the results are interpreted. A positive assay requires antibody complexes to form with both the immobilized viral antigens in the testing well 16 and the control antibody in the control well 18. A negative test will not have antibody complexes that recognize viral proteins but will complex with control antigens. If control complexes do not form, or if there is only binding to viral antigens, the diagnostic assay is deemed invalid.

The assay of the present invention is accessible to determine the impact of FcaGHV1 infection on domestic cat health, largely to assess the role of FcaGHV1 in intestinal lymphomas and other feline malignancies. The assay is a user-friendly, rapid (less than 15 minutes) device that can be deployed to veterinary clinics, hospitals, and rescues to determine seroprevalence of FcaGHV1 in domestic cats.

This device can be manufactured at a low cost to yield an intuitive product that is shelf stable. Serological confirmation of FcaGHV1 infection will lead to changes in point-of-care by both veterinarians and pet owners. Indication of infection will result in more frequent exams and screening, as well as differential approaches in lymphoma/cancer treatment. This product may be sold to existing veterinary clinics, hospitals, and animal organizations, such as rescues and animal control agencies.

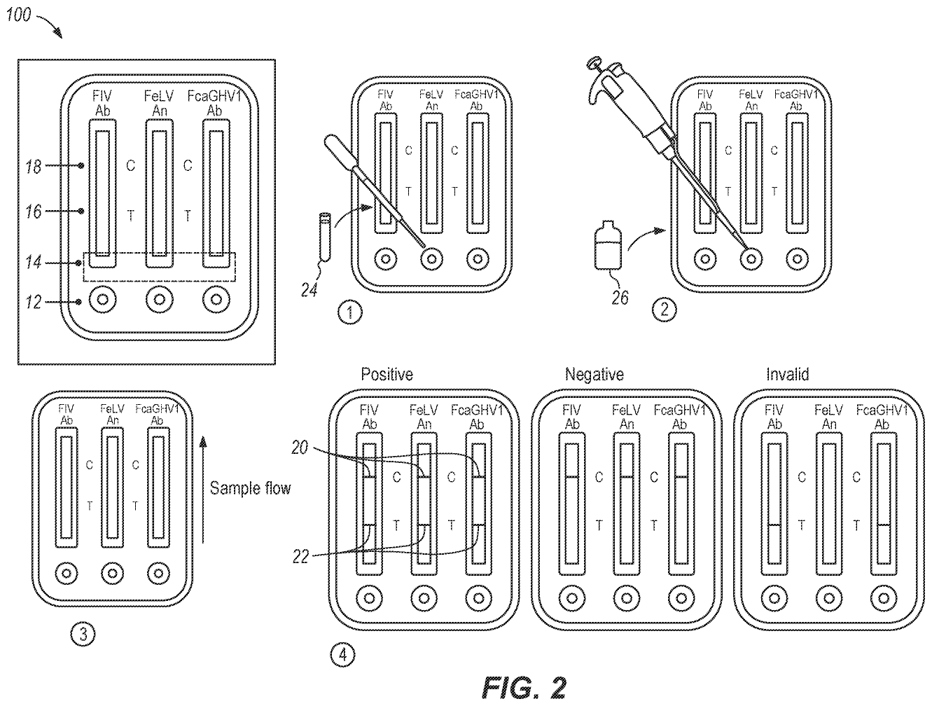

As shown in FIG. 2, an alternative embodiment of the lateral flow assay device 100 of the present invention may be used to detect FcaGHV1, and FeLV, and/or FIV. Devices that determine infection status for FIV and FeLV have been utilized in a veterinary clinical setting for over 20 years in the US. The method for detection of tumor-associated viruses has been well established as the standard of care in feline wellness care. In a proactive veterinary approach, clinicians can utilize one device to detect all viral tumor-associated infections, as shown in FIG. 2. Detection of FIV infection is performed in an antibody lateral flow assay. FIV antigens (p15 and p24) are immobilized on the test well and anti-FIV antibodies in the blood will bind to the test line as with FcaGHV1 detection. Due to vaccination efforts, FeLV infection is detected through an antigen lateral flow assay. In a similar manner to an antibody LFA, detection of viral antigens in the blood requires anti-FeLV antibodies to be immobilized on the membrane in the test well. In this test, gold-conjugated anti-FeLV antibodies will bind to FeLV antigens in the blood in the conjugate pad. As the antigen/antibody-conjugates flow onto the membrane, they will be recognized and bound by the immobilized antibodies in the test well—resulting in a red line.

As shown in FIG. 2, the process for using the lateral flow assay device of the second embodiment is similar to the process illustrated in FIG. 1. In the sample loading of step 1, a drop of blood or serum 24 is added to each sample well(S) 12. Buffer 26 loading is step 2 during which PBS is added to the sample wells 12. During the sample incubation of step 3, capillary action moves the sample across the lateral flow tests as described above. The results are then interpreted for each viral detection assay in step 4. A positive result is indicated by a stripe in the control (C) well 18 and the testing (T) well 16. A negative result is indicated by a stripe in the C well 18 only.

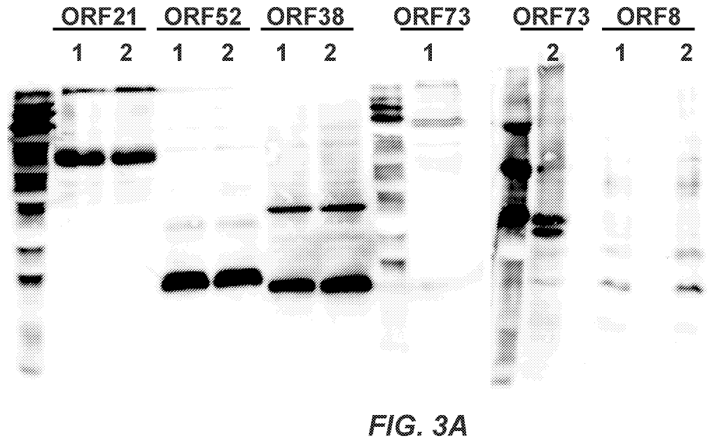

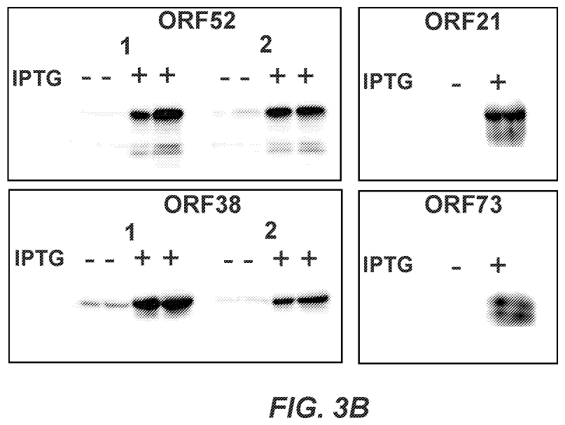

FIG. 3A is a western blot of FcaGHV1 viral antigens 34 derived from eukaryotic cells that are used in the assay. The nucleic acid sequence of FcaGHV1 antigens were optimized from the natural sequence to improve gene expression and increase translational efficiency in human eukaryotic cells for protein purification. HEK293T cells were transiently transfected with 1 ug of pCDNA3.1 encoding ORF21-HIS, ORF52-HIS, ORF38-HIS, ORF73-HIS, or ORF8-HIS using 3 ug of polyethylenimine (PEI). 72 hours post-transfection, cells were harvested in RIPA buffer and lysates were resolved by SDS-PAGE. Viral antigens were detected by immunoblot analysis using an anti-HIS tag antibody.

FIG. 3B is a western blot of FcaGHV1 viral antigens 34 derived from bacteria that are used in the assay. Briefly, BL21 Rosetta E. coli harboring plasmids encoding protein-tagged viral proteins (GST-ORF21, GST-ORF38, GST-ORF52, or GST-ORF73) were induced by vehicle (ddH20) or IPTG and cultured at 20° C. overnight. Cells were harvested in laemmli loading dye and lysates were resolved by SDS-PAGE. Viral antigens were detected by immunoblot analysis using an anti-GST tag antibody.

Further Research: Serology assays only indicate exposure to a pathogen but are not a measure of pathogen-associated disease or burden. GHVs undergo a biphasic infection cycle characterized by an acute phase, frequently called lytic infection, which resolves into a life-long chronic infection called latency. Latency is typically the phase of infection associated with the development of malignant disease in other GHVs infections. Latent infections can reenter the lytic phase of infection following any type of stress to the host, including but not limited to solid-organ-transplants and immunodeficiency. This is of concern for feline infection due to the prevalence of FIV. The rapid serology test will indicate if a cat is infected with FcaGHV1. Serology does not specify the phase of infection or if the cat has infection-associated disease. Further research will need to be conducted to develop clinical assays such as pathology kits for intestinal lymphoma biopsies and an FcaGHV1 antigen test to use in blood/nasal swabs for indication of acute infection.

### REFERENCES

1. Troyer R M, Beatty J A, Stutzman-Rodriguez K R, et al. Novel Gammaherpesviruses in North American Domestic Cats, Bobcats, and Pumas: Identification, Prevalence, and Risk Factors. J Virol. 2014;88 (8). doi:10.1128/jvi.03405-13;
2. McLuckie A J, Barrs V R, Smith A L, Beatty J A. Detection of Felis catus gammaherpesvirus 1 (FcaGHV1) in peripheral blood B- and T-lymphocytes in asymptomatic, naturally-infected domestic cats. Virology. 2016;497. doi:10.1016/j.virol.2016.07.018;
3. Beatty J A, Troyer R M, Carver S, et al. Felis catus gammaherpesvirus 1; a widely endemic potential pathogen of domestic cats. Virology. 2014;460-461 (1). doi: 10.1016/j.virol.2014.05.007;
4. Stutzman-Rodriguez K, Rovnak J, VandeWoude S, Troyer R M. Domestic cats seropositive for Felis catus gammaherpesvirus 1 are often qPCR negative. Virology. 2016;498. doi:10.1016/j.virol.2016.07.027;
5. Rose E C, Tse T Y, Oates A W, et al. Oropharyngeal Shedding of

Gammaherpesvirus DNA by Cats, and Natural Infection of Salivary Epithelium. Viruses. 2022;14 (3). doi:10.3390/v14030566;
6. Magden E, Quackenbush S L, VandeWoude S. FIV associated neoplasms-A mini-review. Vet Immunol Immunopathol. 2011;143 (3-4). doi:10.1016/j.vetimm.2011.06.016;
7. Louwerens M, London C A, Pedersen N C, Lyons L A. Feline lymphoma in the post-feline leukemia virus era. J Vet Intern Med. 2005;19(3). doi:10.1892/0891-6640(2005)19 [329:FLITPL]2.0.CO;2;
8. Vail D M, Moore A S, Ogilvie G K, Volk L M. Feline lymphoma (145 cases): proliferation indices, cluster of differentiation 3 immunoreactivity, and their association with prognosis in 90 cats. Journal of veterinary internal medicine/American College of Veterinary Internal Medicine. 1998;12(5). doi:10.1111/j.1939-1676.1998.tb02134.x;
9. Beatty J. Viral causes of feline lymphoma: Retroviruses and beyond. Veterinary Journal. 2014;201(2). doi:10.1016/j.tvjl.2014.05.026;
10. Moore P F, Rodriguez-Bertos A, Kass P H. Feline Gastrointestinal Lymphoma: Mucosal Architecture, Immunophenotype, and Molecular Clonality. Vet Pathol. 2012;49(4). doi:10.1177/0300985811404712;
11. Aghazadeh M, Shi M, Pesavento P A, et al. Transcriptome analysis and in situ hybridization for FCAGHV1 in feline lymphoma. Viruses. 2018;10(9). doi:10.3390/v10090464.

The present invention has been described with reference to certain preferred and alternative embodiments that are intended to be exemplary only and not limiting to the full scope of the present invention as set forth in the appended claims.

We claim:

1. A lateral flow assay device for detection of Felis catus gammaherpesvirus 1 (FcaGHV-1), comprising:
a cassette, wherein said cassette comprises a sample well for receiving a drop of blood or serum from a patient cat, wherein said cassette further comprises a testing well and a control well, wherein a result of an assay is displayable in said testing well and a validity of an assay is displayable in said control well;
a conjugate release pad positioned in said cassette, wherein said conjugate release pad comprises a plurality of gold nanoparticle-conjugated anti-feline IgG antibodies for binding IgG antibodies in said drop of said blood or serum from said patient cat;
a membrane positioned in said cassette having a test region and a control region, wherein said test region of said membrane comprises a plurality of FcaGHV-1 antigens for binding antibodies against FcaGHV-1 antigens, wherein said control region of said membrane comprises a plurality of Protein A proteins for binding feline IgG antibodies.

2. The lateral flow assay device of claim 1, further comprising a glass-fiber sample pad.

3. The lateral flow assay device of claim 1, further comprising a cellulose absorbent pad.

4. The lateral flow assay device of claim 1, further comprising a lateral flow assay for detection of feline immunodeficiency virus.

5. The lateral flow assay device of claim 1, further comprising a lateral flow assay for detection of feline leukemia virus.

6. A method of detecting Felis catus gammaherpesvirus 1 (FcaGHV-1) using a lateral flow assay device, comprising the steps of:
supplying a lateral flow assay device comprising a cassette, wherein said cassette comprises a sample well, a testing well, and a control well, wherein a conjugate release pad is positioned in said cassette, wherein said conjugate release pad comprises a plurality of gold nanoparticle-conjugated anti-feline IgG antibodies, wherein a membrane is positioned in said cassette having a test region and a control region, wherein said test region of said membrane comprises a plurality of FcaGHV-1 antigens, wherein said control region of said membrane comprises a plurality of Protein A proteins;
introducing a sample of blood or serum of a patient cat to said sample well of said cassette;
introducing phosphate buffered saline to said sample well to dilute said sample of blood or serum;
binding a plurality of IgG antibodies from said sample of blood or serum to at least some of said plurality of gold nanoparticle-conjugated anti-feline IgG antibodies on said conjugate release pad to form a plurality of complexes;
binding at least some of said plurality of complexes to at least some of said plurality of FcaGHV-1 antigens to display a positive result in said testing well; and
binding at least some of said plurality of complexes to at least some of said of Protein A proteins to display a valid result in said control well.
